(19) United States
(12) Patent Application Publication
(10) Pub. No.: US 2025/0303034 A1
(43) Pub. Date: Oct. 2, 2025

(54) CONTAINER ARRANGEMENT FOR THE PREPARATION AND SUPPLY OF AN ALKALIZING SOLUTION, AND EXTRACORPOREAL BLOOD TREATMENT MACHINE WITH THE CONTAINER ARRANGEMENT

(71) Applicant: B. Braun Avitum AG, Melsungen (DE)

(72) Inventors: Thorsten Hage, Bad Iburg (DE); Dinah Fiebig, Muenster (DE)

(21) Appl. No.: 19/083,766

(22) Filed: Mar. 19, 2025

(30) Foreign Application Priority Data
Mar. 26, 2024 (DE) 10 2024 108 582.7

(51) Int. Cl. A61M 1/14; A61M 1/16

(52) U.S. Cl. CPC A61M 1/1566; A61M 1/1565; A61M 1/1666; A61M 1/1668

(57) ABSTRACT

A container arrangement for an extracorporeal blood treatment machine serves as a replacement for an insert container designed with a predetermined topology for supplying an alkalizing solution. The container arrangement includes a container for holding an alkalizing dry substance. The container has a container intake, a container drain, and an adapter. The adapter has an inflow connection section for coupling and fluidic connection to an inflow of an insert container holder of the extracorporeal blood treatment machine and an outflow connection section for coupling to an outflow of the insert container holder of the extracorporeal blood treatment machine. A solvent flow path can be formed from the inflow connection section of the adapter, via the container intake, through the container, and to the container drain.

### CROSS-REFERENCE TO RELATED APPLICATION

This application claims priority under 35 U.S.C. § 119 to German Application No. 10 2024 108 582.7, filed on Mar. 26, 2024, the content of which is incorporated by reference herein in its entirety.

### FIELD

The present disclosure relates to a container arrangement for preparing and supplying an alkalizing solution of alkalizing dry substance for an extracorporeal blood treatment machine, and to an extracorporeal blood treatment machine, in particular a dialysis machine, for an extracorporeal blood treatment, such as a hemodialysis, a hemofiltration, a hemodiafiltration, and/or an ultrafiltration. The blood treatment machine has a dialyzer with a semipermeable membrane for a mass transfer between a patient's blood carried in an extracorporeal blood circuit and a dialysis fluid of a dialysis fluid circuit which passes through the dialyzer via a blood inlet and a blood outlet of the dialyzer. A mixing unit of the blood treatment machine mixes ultrapure water with an alkalizing dry substance, in particular with a bicarbonate dry substance, in particular to prevent possible acidosis. An acidic concentrate is also added in the mixing unit. The fresh dialysis fluid produced in this way is then supplied to the dialysis fluid circuit.

### BACKGROUND

In an extracorporeal blood treatment, for example blood purification in the form of hemodialysis, hemofiltration, or hemodiafiltration, blood is removed from a dialysis patient via an arterial vascular access and fed to a dialyzer for blood treatment via an extracorporeal blood circuit. The dialyzer is also supplied with fresh dialysis fluid produced as required via a dialysis fluid circuit. To produce the fresh dialysis fluid, ultrapure water is provided by a water treatment system, in particular a reverse osmosis system, degassed and then mixed with an alkalizing dry substance and an acidic concentrate in a mixing unit. Bicarbonate, for example, is used as an alkalizing dry substance. In the case of intermittent or outpatient blood treatment, particularly in the treatment of chronic renal insufficiency, the bicarbonate is provided in an insert container that has a predetermined size and connection topology adapted to the blood treatment machine. An insert container holder of predetermined size and connection topology is provided on the blood treatment machine. The insert container is inserted into this specially adapted insert container holder. A sensor system is used to detect whether both connections of the insert container holder—i.e., an inflow connection where the ultrapure water is present and an outflow connection where the mixed bicarbonate solution is provided—are actually coupled to the holder and fluidically connected. This detects whether the insert container holder is actually filled with the predetermined insert container and whether the necessary fluid connection of the inflow and outflow is correctly formed so that the bicarbonate dry substance can be dissolved reliably and as intended. If the insert container holder is detected as correctly filled, a control unit releases the mixing mode, which is itself a prerequisite for blood treatment.

Conventional insert containers, such as the bicarbonate cartridge distributed under the federally registered trademark SOL-CART® B, which originated with the applicant, or the concentrate bag distributed under the federally registered trademark BIBAG®, contain a comparatively small amount of dry substance and only ever support a one-off blood treatment.

The disadvantage of these solutions is that the small quantity requires frequent conversion/replacement of the insert container. In addition, these insert containers are intended for single use only and any unused quantities must be discarded. This means that the use of primary packaging materials, in particular the plastic used for the insert container, such as PET, PE, or PP, is high, which has a negative impact on environmental compatibility/balance.

### SUMMARY

The object of the present disclosure, on the other hand, is to avoid or at least minimize the disadvantages of the prior art and, in particular, to provide a container arrangement and an extracorporeal blood treatment machine which provides a more efficient and safer intermittent or ambulatory extracorporeal blood treatment.

The problem of the present disclosure is solved with respect to a container arrangement according to disclosure and with respect to the extracorporeal blood treatment machine according to disclosure.

A basic idea of the present disclosure provides, for the supply of an alkalizing dry substance and for the production and supply of an alkalizing solution, to insert, mechanically couple, and fluidically connect an adapter adapted according to this topology into an insert container holder of an extracorporeal blood treatment machine designed with a predetermined mechanical and fluidic connection topology, instead of an insert container predeterminedly designed according to this topology. The adapter can have smaller dimensions in terms of volume compared to the predetermined insert container. Apparently, a container containing the alkalizing dry substance and designed independently of the topology of the insert container holder is fluidically connected to this adapter. In particular, the container can be provided at a distance from the insert container holder on the extracorporeal blood treatment machine.

The advantage is that at least the solvent inflow, and preferably also the solution outflow of the insert container holder, of the blood treatment machine can be used by the adapter to supply solvent/ultrapure water and preferably to provide the alkalizing solution, and that the amount of dry substance provided by the container is no longer limited to the comparatively small amount of the predetermined insert container. Preferably, the container of the container arrangement according to the disclosure is designed and configured for use over several blood treatments, in particular with regard to its material and its content quantity. With this container, which preferably contains a larger quantity of dry substance than the predetermined insert container, several blood treatments can thus be carried out on the same blood treatment machine in sequential order, one can also say continuously, without having to change the container. Compared to the predetermined, comparatively smaller insert container, the comparatively larger container fluidically connected via the adapter must therefore be changed less often, which leads to reduced effort during blood treatments and to a cost reduction in the manufacturing process, since fewer containers/container material are/will be required for a predetermined amount of dry substance. In addition, unlike the insert container, which is intended to be used as a single-use container, there is no regular disposal of residual quantities. As an alternative to the solution according to the disclosure, the insert container holder can of course continue to be used with the predetermined insert container in single use.

In other words, an underlying idea of the present disclosure is to provide an existing/known extracorporeal blood treatment machine, instead of a predetermined insert container—which is predetermined in particular with regard to its contained amount of dry substance and its connection topology and connection dimension, in particular the topology and dimensions of its fluid inflow and outflow—to provide an adapter with the same predetermined connection topology and connection dimensions and to fluidically connect a container with a preferably larger quantity of dry substance compared to the predetermined insert container to the adapter. The adapter according to the disclosure is preferably provided and designed to be inserted into a predeterminedly designed insert container holder of the existing/known extracorporeal blood treatment machine provided for the predeterminedly designed insert container. Thus, on the one hand, the supply of solvent, preferably ultrapure water, can obviously take place via the predetermined insert container holder, and on the other hand, a container containing the dry substance can be connected/used, which is freely selectable in terms of its contained quantity, its connection topology, and its dimensions, in particular the topology and dimensions of its fluidic inflow and outflow.

In yet other words, according to the present disclosure, a container arrangement is provided for one or an extracorporeal blood treatment machine which is intended and designed for intermittent or ambulatory blood treatment. The container arrangement according to the disclosure is provided and designed as a replacement for an insert container designed with a predetermined topology for supplying an alkalizing dry substance, for dissolving the dry substance, and for supplying the resulting alkalizing solution, in particular a bicarbonate solution. The container arrangement has the following features:
a container which is intended and designed to hold the alkalizing dry substance, to bring it into contact with a solvent, and to provide the alkalizing solution, the container having a container intake which is intended and designed for fluidic connection to a solvent inflow of the blood treatment machine, and a container drain which is intended and designed to supply the alkalizing solution, in particular to a dialysis fluid circuit of the extracorporeal blood treatment machine;
an adapter, in particular a fluidic adapter, which is intended and designed to be inserted as a replacement for the insert container into a predetermined insert container holder of the extracorporeal blood treatment machine. Preferably, the insert container holder is provided for coupling and fluidic connection with the insert container and has a predetermined design. For this purpose, the insert container holder preferably has an inflow that can be fluidically connected, in particular connected, to the solvent inflow of the extracorporeal blood treatment machine and an outflow that can be fluidically connected, in particular connected, to the dialysis fluid circuit of the extracorporeal blood treatment machine. The inflow and outflow of the insert container holder are preferably arranged and designed in a predetermined way in relation to each other. In particular, they are arranged and designed in a predetermined way in relation to each other in accordance with the predetermined topology of the insert container. The adapter apparently has an inflow connection section, which is provided and designed for coupling and fluidic connection with the inflow of the insert container holder of the extracorporeal blood treatment machine, and an outflow connection section, which is provided and designed at least for coupling—that is, for positioning and/or fastening, but not necessarily also for fluidic connection—with the outflow of the insert container holder of the extracorporeal blood treatment machine;
and at least one solvent flow path, preferably a solvent line and/or a solvent tube, which is provided and can be formed from the inflow connection section of the adapter, via the container intake, through the container and to the container drain.

As already mentioned above, the container arrangement according to the disclosure makes it possible to use the predetermined insert container holder of an extracorporeal blood treatment machine for the fluidic connection of a container which does not fulfill/does not have to fulfill the topology of the predetermined insert container. With the aid of the container arrangement according to the disclosure, different containers with different topologies, in particular different connection topologies, can therefore be coupled and fluidically connected to the insert container holder. This is due to the fact that it is not the container that is inserted and coupled into the insert container holder, but the adapter of the container arrangement, which has the topology required by the insert container arrangement, in particular the connection topology. A container of any topology and any volume can therefore be fluidically connected. As mentioned above, the predetermined insert container is intended for single use. Unused residual quantities of the contained dry matter must then always be discarded. With the container arrangement according to the disclosure, even large containers can be connected as containers that contain significantly more dry substance than the predetermined insert container. This in turn supplements the use of the predetermined insert container on the extracorporeal blood treatment machine with containers of any size and topology, so that the connected container can be used beyond the one-time performance of the blood treatment/dialysis therapy. The frequent retooling/replacement that is necessary when using the comparatively small, predetermined insert container, regardless of its residual fill level after each therapy, is reduced or is no longer necessary. By means of the container arrangement according to the disclosure, multiple applications are thus possible with a correspondingly large container, for example with a content of more than one liter, in particular two to five liters of dry substance, and the content can be used over several blood treatments. Any surplus from a first blood treatment is thus still available for a subsequent blood treatment and does not have to be discarded. A new container with alkalizing dry substance must only be connected to the adapter once the dry substance in the container has been used up. This significantly reduces the amount of primary packaging materials used, in particular the plastic used for the container, such as PET, PE, or PP. This also significantly improves the environmental compatibility/balance of the product.

In short, a container arrangement for supplying alkalizing dry substance and its solution is provided according to the disclosure, by which intermittent or ambulatory extracorporeal blood treatment can be performed more efficiently and safely.

Preferably, the container/the amount of dry substance contained is dimensioned so that the blood treatment can be carried out continuously over a week using the blood treatment machine. The volume of the container/the amount of dry substance contained is more than one liter, in particular 1.5 to 5 liters.

Since the container does not (or no longer) have to fit topologically into the predetermined insert container holder, it is preferably optimized with regard to its storage and/or transport. Preferably, it has a cuboid, in particular stackable, basic shape.

The alkalizing dry substance is preferably a pharmaceutical solid concentrate, preferably a dialysis concentrate, based on a bicarbonate, preferably sodium bicarbonate, for dynamic multiple application in the course of dialysis therapy.

According to a possible further development of the container arrangement, a first supply flow path starting from the container drain and bypassing the outflow connection of the adapter is provided for supplying the alkalizing solution produced in the container by dissolving the dry substance to the dialysis fluid circuit of the extracorporeal blood treatment machine. In this way, the container drain can be connected independently of the outflow connection of the adapter and therefore independently of the outflow of the insert container holder. This allows very flexible provisioning, for example to a solution container.

Preferably, the first supply flow path opens/ends at a withdrawal lance that can be inserted/immersed in a solution container of the extracorporeal blood treatment machine.

In an alternative or supplementary further development of the container arrangement, a second supply flow path from the container drain to the outflow connection of the adapter is provided for supplying the alkalizing solution produced in the container by dissolving the dry substance to the dialysis fluid circuit of the extracorporeal blood treatment machine. In this way, the container drain can be connected via the outflow connection of the adapter and using the outflow of the insert container holder. Provision can thus take place via the predetermined fluidic connection of the outflow of the insert container holder of the extracorporeal blood treatment machine.

According to a preferred further development, the container arrangement has a bypass flow path, preferably switchable, which is provided from the solvent connection of the adapter to the outflow connection of the adapter, bypassing the container. The bypass flow path preferably runs inside the adapter, preferably as a tube or pipe connection. In this way, the container of the container arrangement containing the dry substance can be switched to the bypass or bypassed at any time, and the adapter enables the lines and tubes of the extracorporeal blood treatment machine to be disinfected without the container having to be removed or dismantled. Despite the preferably large amount of dry substance in the container, it can be disinfected at any time without having to remove or dismantle the container.

In order to be able to switch at least the above-mentioned solvent flow path, in a preferred further development the container arrangement has a first directional control valve with switching positions downstream of the inflow connection section. The solvent flow path is opened in a first switching position of the first directional control valve and closed in a second switching position of the first directional control valve. In the simplest version, the first directional control valve is designed as a manually operated 2/2-way valve with two connections and two switching positions, wherein one of the connections is fluidically connected to the inflow connection section and the other to the container intake.

In order to be able to switch the above-mentioned second supply flow path via the container drain to the outflow connection section of the adapter, in a preferred further development the container arrangement has a second directional control valve with switching positions upstream of the outflow connection section of the adapter. The second supply flow path is opened in a first switching position of the second directional control valve and closed in a second switching position of the second directional control valve. In the simplest version, the second directional control valve is also designed as a manually operated 2/2-way valve with two connections and two switching positions, wherein one of the connections is fluidically connected to the container drain and the other to the outflow connection section of the adapter.

In the case of the further development with the bypass flow path, this can preferably be switched by means of the first and second directional control valves. Preferably, the bypass flow path is closed with the first switching positions of the two directional control valves and opened with the second switching positions of the two directional control valves. In this case, the directional control valves are preferably designed as manually operated 3/2-way valves with three connections and two switching positions. The 3/2-way valves have the same ports and connections as the two 2/2-way valves mentioned above, but are each supplemented by a third port, wherein the third ports are connected to each other via the bypass flow path.

The solvent flow path can be routed differently within the container, resulting in different structural designs or basic concepts of the container.

According to a first further development of the container, both the container intake, at which an inlet of the solvent is provided, and the container drain, at which the outlet of the alkalizing solution is provided, are provided at the highest point or region of the container. For this purpose, a dip tube extends from the container drain into the container and discharges at the lowest point or region of the container. The dip tube preferably has a filter at this opening to prevent undissolved dry matter from entering. The solvent thus enters the top of the container and flows/seeps through the dry substance and dissolves it, so that the alkalizing solution enters the dip tube at the lowest point or region of the container and is displaced to the container drain, in particular due to the continuously flowing solvent.

In an alternative embodiment, the container intake is at the highest point or region of the container and the container drain is provided on the edge of the container, wherein in this case, too, a dip tube extends from the container drain into the container and opens into the lowest point or region of the container. In this case too, the mouth of the dip tube preferably has a filter to prevent undissolved dry matter from entering the dip tube.

In an alternative embodiment, the container intake is provided at the highest point or region of the container and the container drain is provided at the lowest point or region of the container. In this case, a filter is preferably installed upstream of the container drain to prevent undissolved dry matter from escaping.

According to the present disclosure, an extracorporeal blood treatment machine, in particular a dialysis machine, is provided and designed for the intermittent or ambulatory extracorporeal blood treatment of a patient's blood. Obviously, it shows:
a dialyzer;
a dialysis fluid circuit which runs through the dialyzer via a dialysis fluid inlet and a dialysate outlet of the dialyzer;
a mixing unit which is provided and adapted for mixing/dissolving at least ultrapure water and an alkalizing dry substance, preferably a bicarbonate dry substance, preferably a sodium bicarbonate, to form an alkalizing solution and supplying the alkalizing solution to the dialysis fluid circuit;
an insert container holder of the mixing unit, which is designed according to at least one aspect of the preceding description, and which is provided and designed for coupling and fluidic connection to an insert container designed with a predetermined topology for providing the alkalizing solution, wherein the insert container holder has an inflow which is fluidically connectable, in particular connected, to a solvent inflow of the extracorporeal blood treatment machine, and has an outflow which is fluidically connectable, in particular connected, to the dialysis fluid circuit for providing the alkalizing solution. The inflow and outflow of the insert container holder are apparently arranged and designed in a predetermined manner in relation to one another, in particular in accordance with a predetermined topology of the insert container to be held by the insert container holder and;
a container arrangement embodied in accordance with at least one aspect of the preceding description, the adapter of the container arrangement being inserted into the insert container holder, preferably in place of the predetermined insert container, the inflow connection section of the adapter being coupled to and fluidically connected to the inflow of the insert container holder, and the outflow connection section of the adapter is at least coupled to the outflow of the insert container holder, and wherein at least the solvent flow path from the inflow connection section of the adapter, via the container intake of the container, through the container and to the container drain is provided and can be formed, in particular is formed.

The advantages of this revelational extracorporeal blood treatment machine have been explained in detail in the course of the description of the revelational container arrangement, so that reference is made to the above description of the advantages in order not to overload this writing. In short, an extracorporeal blood treatment machine is provided for intermittent or outpatient extracorporeal blood treatment, with which the blood treatment can be carried out more efficiently and safely.

According to a preferred training, the extracorporeal blood treatment machine has a detection unit, in particular a sensor unit, which is at least adapted to detect whether or not the couplings of the inflow connection section of the adapter with the inflow of the insert container holder and the outflow connection section of the adapter with the outflow of the insert container holder are formed. In other words, the detection unit can at least detect whether the adapter is correctly inserted into the insert container holder or not. The detection unit is adapted to output a signal dependent on a result of the detection, wherein a control unit of the blood treatment machine is connected by signal to the detection unit and is adapted to open a fluid connection of the solvent inflow with the inflow of the insert container holder only when at least the two couplings are detected as being formed, and to close them when only one or neither of the couplings is detected as being formed.

The opening/closing of the fluidic connection between the solvent inflow and the inflow of the insert container holder is preferably done by activating a check valve located between the solvent inflow and the inflow.

It is particularly preferred that the detection unit additionally be adapted to detect whether or not the fluidic connection of at least the inflow connection section of the adapter is formed with the inflow of the insert container holder.

Alternatively or additionally, at least the inflow connection section, and preferably also the outflow connection section, is designed in such a way that, when correctly coupled to the inflow of the insert container holder, it automatically forms the fluidic connection there correctly, for example, by the inflow connection section/or the outflow connection section, when coupled to the inflow/or to the outflow, pushing open or opening a non-return valve located there, and thus automatically forming the fluidic connection when the adapter is inserted.

According to a preferred further training of the extracorporeal blood treatment machine, the first supply flow path mentioned above from the container drain, bypassing the outflow connection section of the adapter, to the dialyzing fluid circuit is intended and can be designed to provide the alkalizing solution to the dialyzing fluid circuit, in particular it is designed to do so. For this purpose, the container drain is preferably connected by means of a pipe or a tube to a sampling lance, which is inserted or immersed in particular in a solution container of the extracorporeal blood treatment machine, which is provided for holding and supplying the solution.

In accordance with a preferred alternative or supplementary training course for the extracorporeal blood treatment machine, the second supply flow path mentioned above is intended and can be designed to provide the alkalizing solution to the dialyzing fluid cycle from the container drain, via the outflow connection section of the adapter, the outflow of the insert container holder and to the dialysis fluid circuit is provided and can be formed, in particular is formed. For this purpose, the container drain is preferably connected to the outflow connection section of the adapter by means of a pipe or tube. In the case of the second supply flow path, the outflow connection section of the adapter is preferably not only coupled to the outflow of the insert container holder, but also fluidically connected to it, so that the alkalizing solution can be provided to the dialysis fluid circuit via this outflow.

As already mentioned above, the bypass flow path from the inflow connection section of the adapter to the outflow connection section of the adapter, bypassing the container, is preferably designed and can be formed, in particular.

As already explained above, the first directional control valve with switching positions is preferably provided downstream of the inflow connection section of the adapter, in accordance with a further development of the extracorporeal blood treatment machine, wherein the solvent flow path is opened in the first switching position of the first directional control valve and is closed in the second switching position of the first directional control valve.

As already explained above, the second directional control valve with switching positions is preferably provided in accordance with a further development of the extracorporeal blood treatment machine upstream of the outflow connection section of the adapter, the second supply flow path being opened in the first switching position of the second directional control valve and closed in the second switching position of the second directional control valve.

As already explained above, the preferred method is to close the bypass flow path with the first switching positions of the first and second directional control valves and to open it with the second switching positions of the first and second directional control valves in accordance with a further development of the extracorporeal blood treatment machine.

### BRIEF DESCRIPTION OF THE DRAWINGS

The disclosure will be explained in more detail below by means of preferred embodiments, with the aid of figures. The following is shown:

FIG. 1 shows a schematic, fluidic diagram of an extracorporeal blood treatment machine according to a preferred embodiment;

FIG. 2 shows a container arrangement according to a first embodiment for the extracorporeal blood treatment machine according to FIG. 1;

FIG. 3 shows a container arrangement according to a second embodiment, which is installed in the extracorporeal blood treatment machine according to FIG. 1;

FIG. 4 shows a container arrangement according to a third embodiment for the extracorporeal blood treatment machine according to FIG. 1;

FIG. 5 shows a container arrangement according to FIG. 4 in a bypass circuit; and

FIG. 6 through 8 show three containers of the container arrangement according to three embodiments.

The Figures are schematic in nature and are intended only to aid understanding of the revelation. Identical elements are marked with the same reference signs. Features of different designs can be exchanged among themselves.

### DETAILED DESCRIPTION

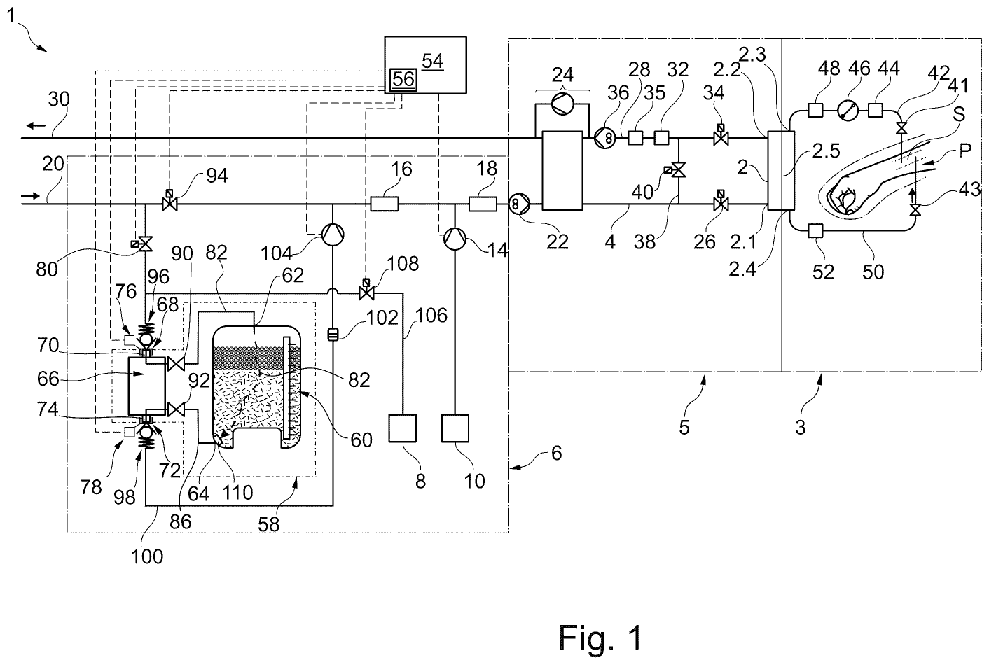

FIG. 1 shows a schematic view of a fluidic circuit diagram of an extracorporeal blood treatment machine 1 (referred to in the following only as blood treatment machine) in the form of a dialysis machine for intermittent, in particular outpatient, extracorporeal blood treatment of blood of a patient P according to a preferred embodiment of the present disclosure.

In particular, the design and use of a container arrangement 58 of the blood treatment machine 1 according to the disclosure are described, with the aid of which an alkalizing dry substance provided for intermittent, in particular outpatient, extracorporeal blood treatment, is dissolved and the alkalizing solution thus produced is provided to a dialyzing fluid circuit 5 of the blood treatment machine 1.

According to the description, at least the provision and the dissolving of the alkalizing dry substance, as well as the provision of the alkalizing solution for blood treatment machine 1, do not take place in a centralized manner, but rather at blood treatment machine 1 itself. By contrast, the provision and dissolution of an acidic, saline, or other dry substance, as well as the provision of the corresponding solution for blood treatment machine 1, can be carried out in a centralized manner in larger mixing units, for example for several blood treatment machines 1, which is particularly justified by the fact that, for example, acidic solutions are antimicrobial in themselves and can be stored in large quantities and thus for long periods of time. Alkalizing solutions, on the other hand, are to be stored in smaller quantities and close to the time of extracorporeal blood treatment, which leads to the above-mentioned “non-centralized” provision in smaller quantities.

The blood treatment machine 1 has, as shown in FIG. 1, a dialyzer 2 as a central component with, on the one hand, a dialyzing fluid inlet 2.1 and dialysate outlet 2.2 on the dialyzing fluid side and, on the other hand, a blood inlet 2.3 and blood outlet 2.4 on the blood side of an extracorporeal blood circulation 3. Inside, the dialyzer 2 is divided into a dialysis fluid side and a blood side by means of hollow fibers of a semipermeable membrane 2.5.

The dialyzing fluid inlet 2.1 can be connected to a mixing unit 6 via a dialyzing fluid inflow 4, in particular it is connected. This continuously produces fresh dialysis fluid from at least partially degassed ultrapure water, an alkalizing dry substance, and an acidic concentrate.

As an option for providing an alkalizing and an acidic concentrate, the mixing unit 6 has a first and second solution container 8, 10, in which a ready alkalizing and ready acidic solution are respectively provided, as well as a first and second conveying device 12, 14 and, downstream of the conveying devices 12, 14, a first and second measuring device 16, 18 respectively.

According to FIG. 1, the blood treatment machine 1 has a solvent/ultrapure water inflow 20, from which at least partially degassed ultrapure water is continuously supplied by an internal degassing unit (not shown). The ultrapure water inflow 20 can be connected, in particular is connected, via a check valve 94, which is signal-connected to a control unit 54 of the blood treatment machine 1, in fluid communication with the measuring devices 16, 18, a conveying device 22, and a balancing device 24 arranged downstream in series. On the outlet side, the balancing device 24 can be connected, and in particular is connected, to the dialyzing fluid inlet 2.1 of the dialyzer 2 via a dialyzing fluid inflow 4, a valve 26 for shutting off the dialyzing fluid inlet 2.1 being arranged in the dialyzing fluid inflow 4.

The dialysate outlet 2.2 is fluidically connectable, in particular connected, via a dialysate outflow 28 to a disposal outlet 30 for used dialyzing fluid/dialysate. In the dialysate outflow 28, the following are arranged in series in terms of fluidics between the dialysate outlet 2.2 and the disposal outlet 30: an actuatable valve 34 for shutting off the dialysate outlet 2.2, a detection unit 32 for detecting a component in the dialysate, and a fourth conveying device 36, via which the dialysate is conveyed to the balancing device 24 and to the disposal outlet 30 for dialysate. The balancing device 24 ensures that a desired volume of excess water can be removed from the patient's blood by means of an ultrafiltration pump as part of an ultrafiltration process. Upstream of the fourth conveying device 36, a pressure-sensing unit 35 is provided in the dialysate outflow 28 to measure the dialysate outlet pressure.

In addition, a bypass flow path 38 is provided, via which the dialysis fluid inflow 4 can be connected to the dialysate outflow 28. A valve 40 that can be actuated is arranged in the bypass flow path 38, via which the bypass flow path 38 can be closed.

On the blood side, the extracorporeal blood circuit 3 is designed to take blood from the patient via an arterial tubing section 42 and supply it to the dialyzer 2 via the blood inlet 2.3. In the arterial tubing section 42, in the direction of flow, there is an arterial tube clamp 41, an arterial hematocrit or HCT sensor 44, a blood pump 46, and a blood inlet pressure sensor 48. After the patient's blood has been passed through the extracorporeal blood circuit 3, it is removed from the blood side of the dialyzer 2 at the blood outlet 2.4 and fed to the shunt S via a venous tubing section 50. A blood outlet pressure sensor 52 and a venous tube clamp 43 are arranged in the venous tubing section 50. In the dialyzer 2, the blood is passed over the dialysis fluid in a countercurrent flow, and waste products and excess water are removed. The cleaned blood is then returned/restored to the patient P.

According to FIG. 1, the extracorporeal blood treatment machine 1 has a container arrangement 58 for preparing and supplying an alkalizing solution to the dialyzing fluid circuit 5. The container arrangement 58 according to the disclosure can be provided in addition to or instead of the container 8 with the already prepared/premixed alkalizing solution.

From the ultrapure water inflow 20, an inflow path branches off upstream of the check valve 94, in which a check valve 80 that is operable by the control unit and a spring-loaded non-return valve 96 that closes in the direction of flow are arranged. The inflow path ends in a holder 68 of an insert container holder 68, 72 of the mixing unit 6. Diametrically opposed to the holder 68, the insert container holder 68, 72 has an outflow 72. A non-return valve 98 is provided on this in a mirror-image arrangement and with the opposite closing direction and spring loading.

At the outflow 72 of the insert container holder 68, 72, a supply path 100 starts, which, via a filter 102 and a controllable conveying device 104 that is signal-connected to the control unit 54, opens into the flow path coming from the ultrapure water inflow 20 downstream of the check valve 94 and upstream of the first measuring device 16.

A supply path 106, which runs via a check valve 108 that is operable and signal-connected to the control unit 54, leads from the container 8, from which the ready-mixed alkalizing solution can already be provided, and opens into the inflow path between the check valve 80 and the non-return valve 96.

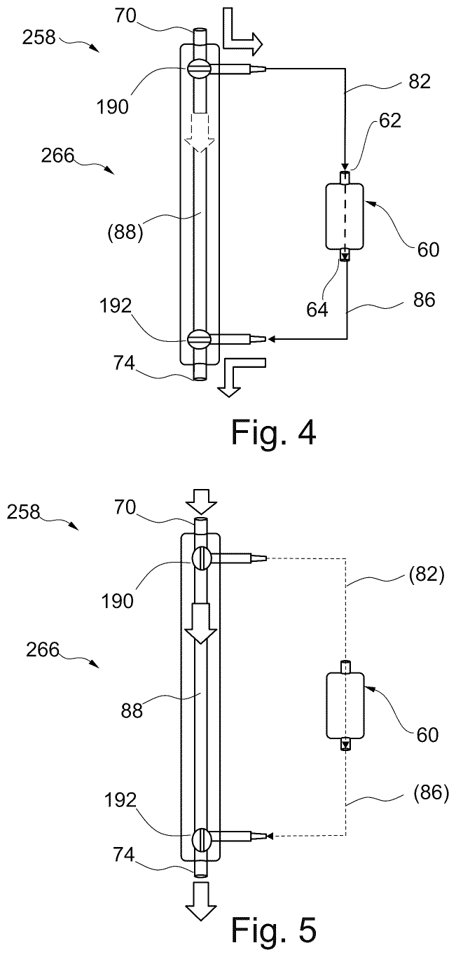

The container arrangement 58 according to the disclosure in FIG. 1 has as a first component an adapter 66 with an inflow connection section 70 and an outflow connection section 74. As a second component, it has a container 60 containing the alkalizing dry substance, with a container intake 62 and a container drain 64. As shown in FIG. 4, the solvent is already in container 60 and is/was supplied to the insert container holder 68, 72 via the inflow 68 (as will be described in more detail below). In addition, the container arrangement 58 according to FIG. 1 has a solvent flow path 82, via which the inflow connection section 70 of the adapter 66 is fluidically connected to the container intake 62. A manually operable first directional control valve 90, configured as a 2/2-way directional control valve, is arranged in the solvent flow path 82, via which valve the container intake 62 can be connected or disconnected in terms of fluid from the inflow connection section 70 of the adapter 66, depending on the switching position.

The solvent flow path 82 extends beyond the container intake 62, through the container 60 and the dry substance/solution therein, to the container drain 64, where a filter 110 is installed upstream to retain undissolved dry substance.

The container drain 64 can be fluidically connected, in particular connected, to the outflow connection section 74 of the adapter 66 via a second provision path 86, wherein, in the second provision path 86—in particular in analogy to the first section of the solvent flow path 82 from the inflow connection section 70 to the container intake-a manually operable second directional control valve 90 in the form of a 2/2-way directional control valve is arranged, via which the container drain 64 can be fluidically connected to or disconnected from the outflow connection section 74 of the adapter 66, depending on the switching position.

As shown in FIG. 1, the container arrangement, consisting of adapter 66, container 60, solvent flow path 82, second supply path 86, and the two directional control valves 90, 92, is properly connected to the insert container holder 68, 72. This means that the connection sections 70, 74 of the adapter 66 are correctly coupled with their associated holders 68, 72 of the insert container holder 68, 72. A detection unit 76, 78 connected to the control unit 54 by a signal detects whether the coupling is correctly formed and reports to the control unit 54. In particular, it is reported whether both connection sections 70, 74 are actually coupled correctly with the respective holder 68, 72, or only one or neither. The detection unit 76, 78 can, for example, be formed by a light barrier or a contact switch at each of the holders 68, 72, which transmits a corresponding signal to the control unit 54 when the connection section 70, 74 is correctly coupled.

The insert container holder 68, 72 of the disclosed blood treatment machine 1 has a predetermined connection topology, that is, a predetermined arrangement of the inflow holder 68 relative to the outflow holder 72, as well as preferably predetermined dimensions of the inflow holder 68 and the outflow holder 72.

In this way, it is prepared to hold a specific/predetermined insert container (not shown) of alkalizing dry matter that corresponds to this predetermined connection topology as a counterpart, to couple each with the inflow 68 and outflow 72 and connect them fluidically. The advantage of the predetermined connection topology of the insert container holder 58 is that no container can be held, coupled, and fluidically connected in the insert container holder 58 as a counterpart if it does not fulfill or correspond to the predetermined connection topology of the insert container holder 58.

Conversely, the specific/predetermined insert container, that is, the insert container with its predetermined connection topology, can only be inserted, coupled, and fluidically connected in this and no other insert container holder with a different connection topology.

Both ensure that mix-ups are ruled out.

However, this means that the available, specific predetermined insert container for the alkalizing dry substance is comparatively small and therefore contains little dry substance. The amount is optimized for a single blood treatment to cover even long blood treatments and is therefore equipped with an amount that is relatively small but rarely fully utilized. The insert container is designed as a single-use product. This is associated with frequent changing, a scrap quantity that occurs with almost every change, as well as a considerable amount of plastic waste due to the packaging material that is produced.

In order to eliminate or reduce these disadvantages, the container arrangement according to the disclosure provides the adapter 66, which has a topology adapted to the connection topology of the insert container holder 68, 72, in particular of the inflow connection section 70 and the outflow connection section 72.

Accordingly, when the adapter 66 is inserted into the insert container holder 68, 72, the inflow connection section 70 fits exactly into the inflow 68, where it pushes open the non-return valve 96, and the outflow connection section 74 fits exactly into the outflow 72, where it pushes open the non-return valve 98. Accordingly, the inflow connection section 70 is coupled and fluidically connected to the inflow 68 and the outflow connection section 74 is coupled and fluidically connected to the outflow 72.

The control unit 54 is notified of this state by the detection unit 76, 78, so that it can then control the check valve 80 to open and the check valve 94 to close. In this way, ultrapure water is available at the first directional control valve of the container arrangement 58. Next, an operator can actuate the two directional control valves 90, 92 from their second, closing switching position to their first, open switching position. Another constraint is that the check valve 108 is closed.

In this way, ultrapure water flows from the ultrapure water inflow 20, through the check valve 80, the pushed-open non-return valve 96, the inflow 68, the inflow connection section 70 of the adapter 66, the first directional control valve 90, the container intake 62, and along the solvent flow path 82 through the container 60 and dissolves the sodium bicarbonate contained therein. The bicarbonate solution exits container 60 through filter 110 and container drain 64 and flows through the second supply flow path 86, the opened second directional control valve 92, the outflow connection section 74 of the adapter 66, the pushed-open non-return valve 98, and the supply path 100 to the dialysis fluid circuit 5.

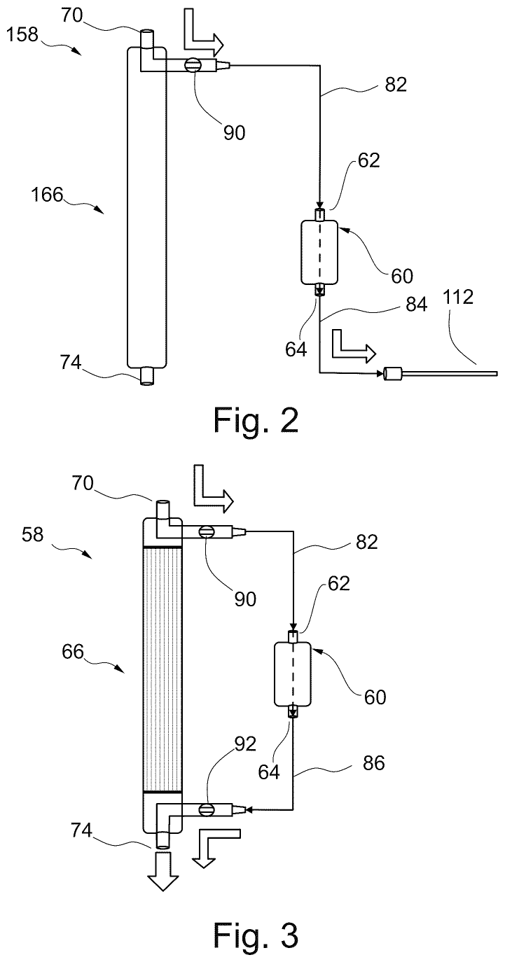

The following FIGS. 2 to 5 show embodiments of a container arrangement 58; 158; 258 according to the disclosure, wherein the embodiment according to FIG. 3 corresponds to the embodiment of the container arrangement 58 shown in FIG. 1.

All embodiments of the container arrangements 58; 158; 258 shown in FIGS. 2 to 5 are suitable for being coupled and fluidically connected to the insert container holder 68, 72 of the blood treatment machine 1 according to FIG. 1, since their respective adapter 66; 166; 266 is configured topologically, in particular with respect to its connection topology, in such a way that it can be inserted with its inflow and outflow connection section 70, 74 into the insert container holder 68, 72 and coupled to the inflow 68 and outflow 72 (see FIG. 1). Depending on the design, the corresponding fluidic connection(s) is (are) then formed.

It should be mentioned at the outset that the container 60 is shown schematically in FIGS. 2 to 5 and is essentially shown only to illustrate the different supply flow paths of the alkalizing solution.

FIG. 2 shows a container arrangement 158 in an embodiment that is comparatively simple. The design of the adapter 166 is such that, as mentioned above, the inflow connection section 70 and the outflow connection section 74 can be inserted into their respective insert container holders 68, 72 (see FIG. 1) and coupled to the associated inflow 68 or outflow 72. However, only the inflow connection section 70 is intended for fluidic connection to the inflow 68. The outflow connection section 74 remains fluidically inactive or “blind” and only fulfills the aforementioned function of occupying the outflow 72 so that the detection unit 76, 78 (see FIG. 1) reports to the control unit 54 that the insert container holder 68, 72 is correctly coupled. Accordingly, the solvent flow path 82 extends from the inflow connection section 70, through the first directional control valve 90, which is designed as a manually operable 2/2-way directional control valve with two switching positions, to the container intake and through the container 60. A first supply flow path 84 extends from the container drain 64, to which a lance 112 is connected. This lance 112 can be inserted into container 8, for example, to fill it. The first directional control valve 90 is switched to its first switching position so that the solvent flow path 82 and the first supply flow path 84 are formed. In its second switching position (not shown), the solvent flow path 82 is closed and no more ultrapure water flows into the container 60.

FIG. 3 shows the embodiment of the container arrangement 58, which is installed in the extracorporeal blood treatment machine according to FIG. 1. Directional control valves 90, 92 are switched to their first switching position so that the solvent flow path 82 and the second supply flow path 86 are formed. In its second switching position (not shown), the solvent flow path 82 is closed, the container 60 no longer supplies ultrapure water, and the outflow connection section 74 no longer supplies alkalizing solution.

FIGS. 4 and 5 show a container arrangement 258 according to a third embodiment. From the inflow connection section 70, a bypass flow path 88 branches off inside the adapter 266, via which the inflow connection section 70 can be connected directly to the outflow connection section 74 of the adapter 266, bypassing the container 60. In order to be able to switch the solvent flow path 82 via the container 60 and the bypass flow path 88, the directional control valves 190, 192 are designed as manually operable 3/2-way directional control valves with three connections and two switching positions, so that in the first switching positions of the directional control valves 190, 192 (see FIG. 4) the bypass flow path is closed and the solvent flow path 82 and the second supply flow path 86 are opened, whereas in the second switching positions (see FIG. 5) of the directional control valves 190, 192, the bypass flow path is opened and the solvent flow path 82 and the second supply flow path 86 are closed. The bypass flow path in the adapter 266 can preferably be used to disinfect the fluidic system from the solvent/high-purity water inflow 20 via the insert container holder 68, 72 and the dialysis fluid circuit 5 by rinsing with a disinfecting fluid, without having to change the container 60. This is simply bypassed during the disinfection (see FIG. 5) and “reactivated” after the disinfection by simply switching the directional control valves 190, 192 to the first switching positions (see FIG. 4).

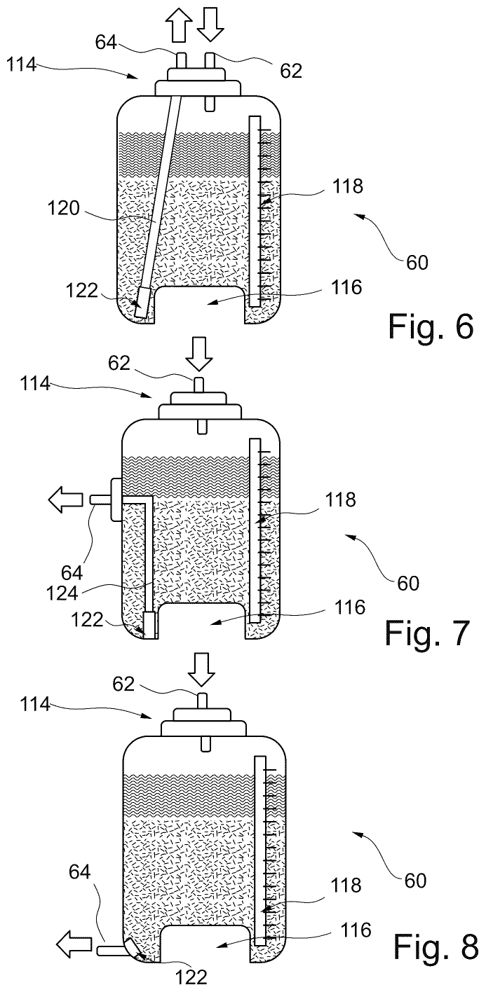

FIGS. 6 to 8 show three embodiments of the container 60, which differ in terms of where the container drain is located and how the solvent flow path is configured within the container.

Common to the embodiments according to FIGS. 6 to 8 is that the container 60 has a cylindrical or cuboid basic shape with rounded edges and/or corners. A screw cap 114 is arranged on one upper side, with the container intake 62, designed as an intake pipe section, passing through it in all cases. The solvent/ultrapure water enters the container 60 through the comparatively short intake pipe 62. Diametrically to the screw cap 114, the container has a recess 116 at the bottom, which is slightly larger than the screw cap 114. This makes it possible to stack several such containers 60, wherein the screw cap 114 of a lower container 60 engages in the recess 116 of the container 60 arranged above it, wherein the stacked containers 60 are fixed in position. The container 60 also has a scale 118 and is made of transparent plastic, for example PE or PET, so that it is always possible to see how much dry substance/dissolved dry substance is still contained in the container 60.

Another effect of the above-mentioned recess 116 is that a ring-shaped, comparatively narrow region is formed in the container 60 around the circumference of the recess 116, which is suitable for the removal of the alkalizing solution to take place here. In all three embodiments of container 60 as shown in FIGS. 6 to 8, this removal occurs at this lowest point or region.

The only difference between the embodiments of container 60 shown in FIGS. 6 to 8 is the arrangement of the container drain 64.

According to FIG. 6, the container drain 64 is designed on the screw cap 114, with a dip tube 120 being provided that extends from the lowest point/region to the screw cap 114 and passes through it. At the lowest point, the opening of the dip tube 120 is covered by a filter 122 to prevent undissolved dry matter from entering the dip tube 120.

According to FIG. 7, the container drain 64 is formed on the edge of the container 60, with a dip tube 124 being provided that extends from the lowest point/region to the edge-side container drain 64. Here, too, an opening of the dip tube 122 is covered at the lowest point by the filter 122 to prevent undissolved dry matter from entering the dip tube 120.

As shown in FIG. 8, the container drain 64 is designed at the edge at the lowest point/region of the container 60, so that a dip tube can be dispensed with. In this case, a filter 122 is installed upstream of the container drain 64 in the container to prevent undissolved dry matter from entering the container drain 64.

List of reference signs

| | |
|---|---|
| 1 | Extracorporeal blood treatment machine |
| 2 | Dialyzer |
| 2.1 | Dialysis fluid inlet |
| 2.2 | Dialysate outlet |
| 2.3 | Blood inlet |
| 2.4 | Blood outlet |
| 2.5 | Semipermeable membrane |
| 3 | Extracorporeal blood circulation |
| 4 | Dialysis fluid inflow |
| 5 | Dialysis fluid circuit |
| 6 | Mixing unit |
| 8 | Container of alkalizing solution |
| 10 | Container of acidic solution |
| 12 | First conveying device |
| 14 | Second conveying device |
| 16 | First measuring device |
| 18 | Second measuring device |
| 20 | Solvent inflow/high-purity water inflow |
| 22 | Conveyor device |
| 24 | Balancing device |
| 26 | First valve |
| 28 | Dialysate outflow |
| 30 | Disposal outlet |
| 32 | Detection unit |
| 34 | Second valve |
| 38 | Bypass flow path (dialysis fluid circuit) |
| 40 | Third valve |
| 41 | Arterial tube clamp |
| 42 | Arterial tubing section |
| 43 | Venous tube clamp |
| 44 | Blood component sensor |
| 46 | Blood pump |
| 48 | Blood inlet pressure sensor |
| 50 | Venous tubing section |
| 52 | Blood outlet pressure sensor |
| 54 | Control unit |
| 56 | Memory |
| 58; 158; 258 | Container arrangement |
| 60 | Container of alkalizing dry matter |
| 62 | Container intake |
| 64 | Container drain |
| 66; 166; 266 | Adapter |
| 68 | Inflow |
| 70 | Inflow connection section |
| 72 | Outflow |
| 74 | Outflow connection section |
| 76, 78 | Detection unit |
| 80 | Check valve |
| 82 | Solvent flow path |
| 84 | First supply flow path |
| 86 | Second supply flow path |
| 88 | Bypass flow path |
| 90; 190 | First directional control valve |
| 92; 192 | Second directional control valve |
| 94 | Check valve |
| 96 | Non-return valve |
| 98 | Non-return valve |
| 100 | Provisioning path |
| 102 | Filter |
| 104 | Conveying device |
| 106 | Deployment path |
| 108 | Check valve |
| 110 | Filter |
| 112 | Lance |
| 114 | Screw cap |
| 116 | Recess |
| 118 | Scale |
| 120 | Dip tube |
| 122 | Filter |
| 124 | Dip tube |
| P | Patient |
| S | Shunt |

1. A container arrangement for an extracorporeal blood treatment machine configured for intermittent or outpatient blood treatment, the container arrangement being a replacement for an insert container designed with a predetermined designed insert container of the extracorporeal blood treatment machine, for providing an alkalizing solution, the container arrangement comprising:
a container;
an adapter; and
a solvent flow path,
the container being configured to hold an alkalizing dry substance, bring the alkalizing dry substance into contact with a solvent, and create the alkalizing solution,
the container comprising a container intake configured for fluidic connection to a solvent inflow of the extracorporeal blood treatment machine and a container drain configured to supply the alkalizing solution,
the adapter comprising an inflow connection section configured for coupling and fluidic connection to an inflow of an insert container holder of the extracorporeal blood treatment machine,
the adapter further comprising an outflow connection section configured at least for coupling to an outflow of the insert container holder of the extracorporeal blood treatment machine, and
the solvent flow path being formable from the inflow connection section of the adapter, via the container intake, through the container and to the container drain.

2. The container arrangement according to claim 1, wherein the container drain is configured to supply the alkalizing solution to a dialyzing fluid circuit of the extracorporeal blood treatment machine.

3. The container arrangement according to claim 2, further comprising a first supply flow path for provision to the dialyzing fluid circuit of the extracorporeal blood treatment machine, starting from the container drain and bypassing the outflow connection section of the adapter.

4. The container arrangement according to claim 3, further comprising a second supply flow path configured to convey the alkalizing solution to the dialyzing fluid circuit of the extracorporeal blood treatment machine, starting from the container drain and leading to the outflow connection section of the adapter.

5. The container arrangement according to claim 4, further comprising a third bypass flow path extending from the inflow connection section of the adapter to the outflow connection section of the adapter bypassing the container.

6. The container arrangement according to claim 5, further comprising a first directional control valve with switching positions, the first directional control valve being located downstream of the inflow connection section of the adapter, the solvent flow path being opened in a first switching position of the first directional control valve and being closed in a second switching position of the first directional control valve.

7. The container arrangement according to claim 6, wherein a second directional control valve having switching positions is provided upstream of the outflow connection section of the adapter, wherein the second supply flow path is opened in a first switching position of the second directional control valve and is closed in a second switching position of the second directional control valve.

8. The container arrangement according to claim 7, wherein:
the third bypass flow path is closed with the first switching position of the first directional control valve and the first switching position of the second directional control valve, and
the third bypass flow path is opened with the second switching position of the first directional control valve and the second switching position of the second directional control valve.

9. The container arrangement according to claim 1, wherein the container intake and the container drain are provided at a highest point or region of the container, a dip tube extending from the container drain into the container and opening into a lowest point or region of the container.

10. The container arrangement according to claim 1, wherein the container intake is provided at a highest point or region of the container and the container drain is provided at an edge, a dip tube extending from the container drain into the container and opening into a lowest point or region of the container.

11. The container arrangement according to claim 1, wherein the container intake is provided at a highest point or region of the container and the container drain is provided at a lowest point or region of the container.

12. An extracorporeal blood treatment machine configured for intermittent or outpatient extracorporeal blood treatment of blood of a patient, the extracorporeal blood treatment machine comprising:
the container arrangement according to claim 1;
a dialyzer;
a dialyzing fluid circuit running through the dialyzer via a dialysis fluid inlet and a dialysate outlet of the dialyzer;
a mixing unit configured to mix at least ultrapure water and an alkalizing dry substance to form an alkalizing solution and to supply the alkalizing solution to the dialyzing fluid circuit; and
an insert container holder,
the insert container holder configured for coupling and fluidic connection to an insert container designed with a predetermined topology for providing the alkalizing solution,
the insert container holder having an inflow that is fluidically connectable to a solvent inflow of the extracorporeal blood treatment machine,
the insert container holder further having an outflow that is fluidically connectable to the dialyzing fluid circuit to provide the alkalizing solution to the dialyzing fluid circuit,
the inflow and the outflow of the insert container holder being arranged and designed in a predetermined manner relative to one another,
the adapter of the container arrangement being inserted into the insert container holder,
the inflow connection section of the adapter being coupled to the inflow of the insert container holder and fluidically connected,
the outflow connection section of the adapter being at least coupled to the outflow of the insert container holder and at least the solvent flow path from the inflow connection section of the adapter, through the container intake of the container, through the container and to the container drain.

13. The extracorporeal blood treatment machine according to claim 12, further comprising a detection unit configured to detect whether or not couplings are formed and to output a signal dependent on whether or not couplings are formed, wherein a control unit of the extracorporeal blood treatment machine, which is signal-connected to the detection unit, is configured to control a fluid connection of the solvent inflow with the inflow of the insert container holder when couplings are detected as being formed, and to close when only one or no coupling is detected as being formed.

14. The extracorporeal blood treatment machine according to claim 13, wherein the control unit closes the fluid connection by activating a check valve arranged between the solvent inflow and the inflow.

15. The extracorporeal blood treatment machine according to claim 12, further comprising a supply flow path for delivering the alkalizing solution to the dialyzing fluid circuit, starting from the container drain and bypassing the outflow connection section of the adapter to the dialyzing fluid circuit.

16. The extracorporeal blood treatment machine according to claim 12, further comprising a supply flow path for supplying the alkalizing solution to the dialyzing fluid circuit, starting from the container drain, via the outflow connection section of the adapter to the dialyzing fluid circuit.
